(19) United States
(12) Patent Application Publication
(10) Pub. No.: US 2012/0179201 A1
(43) Pub. Date: Jul. 12, 2012

(54) Chiropractic spinal manipulation boards

(76) Inventors: Harvey Joe Segur, Portland, OR (US); Sandra Kay Diehl, (US)

(21) Appl. No.: 12/930,459

(22) Filed: Jan. 7, 2011

(51) Int. Cl. A61F 5/00

(52) U.S. Cl. 606/237

(57) ABSTRACT

A portable device that provides a stable rigid platform with two parallel peripheral elevations and a channel in between that can be applied to the spine or pelvis of a patient placed in either a supine, sitting, or standing position, which the doctor uses to obtain therapeutic effects to correct spinal and pelvic disorders, conditions, and syndromes in conjunction with the application of manual manipulation and manipulative surgery beyond what can be done by using the hands alone.

### CROSS-REFERENCED TO RELATED APPLICATIONS

Not Applicable

### FEDERALLY SPONSORED RESEARCH

Not Applicable

### SEQUENCE LISTING OF PROGRAM

Not Applicable

### BACKGROUND

1. Field

This application relates generally to devices that are used in conjunction with the methods, techniques, and procedures of spinal manual manipulation and manipulative surgery by a doctor educated and trained in the professions of either chiropractic, osteopathy, naturopathy, and physical medicine for the treatment of spinal and pelvic pain disorders, conditions, and syndromes.

2. Prior Art

Heretofore, there have been various devices that have been invented that claim to support, brace, stretch, traction, relax, massage, and reposition the articular elements of the spine that have been designed for self-use, self-treatment, or incorporated into a therapeutic table. The concept of the present invention is a portable instrument that provides a stable rigid platform with two parallel peripheral elevations and a channel in between that can be applied to the spine or pelvis of a patient placed in either a supine, sitting, or standing position applied in different locations of the spine and/or pelvis, which the doctor uses to obtain therapeutic effects to correct spinal and pelvic disorders, conditions, and syndromes in conjunction with the application of manual manipulation and manipulative surgery beyond what can be done by using the hands alone.

The prior art thought to be most closely associated with the present invention are the inventions disclosed in U.S. Pat. No. Des. 275,607 to Blackman (1984), Des. 326,913 to Staples (1992), 712,375 to Hartford (1902), 726,054 to Hartford (1903), U.S. Pat. No. 1,398,150 to Pollard (1921), U.S. Pat. No. 1,833,426 to Knudson (1930), U.S. Pat. No. 1,934,918 to Everson (1931), U.S. Pat. No. 2,159,654 to Catlin (1936), U.S. Pat. No. 2,818,854 to Johnson (1958), U.S. Pat. No. 4,230,099 to Richardson (1980), U.S. Pat. No. 4,785,801 to Speece (1988), U.S. Pat. No. 5,007,414 to Sexton (1991), U.S. Pat. No. 6,036,719 to Melius (2000), and U.S. Pat. No. 6,110,194 to Saber (2000). The Blackman design of 1984 does not provide a stable planar base, has a shallow linear arcuate groove, and does not provide for different shapes, sizes, or configuration so it cannot be used in conjunction with manual manipulation. The Staples design of 1992 does not have a stable planar base, so it cannot be used in conjunction with manual manipulation nor does it have a specific stipulated use other than a back bridge. The Hartford invention of 1902 does not demonstrate parallel arcuate peripheral elevations in it's design. The Hartford invention of 1903 does not demonstrate parallel arcuate peripheral elevations in it's design. The Pollard invention of 1921 does not demonstrate parallel arcuate peripheral elevations in it's design. The Knudson invention of 1930 is designed to be used primarily for massage purposes utilizing parallel elevations with multiple rounded projections which is preferably mounted vertical on a wall. The Everson invention of 1931 is designed as a pelvic stabilizer for use with cadavers and not for therapeutic purposes on a live patient. The Catlin invention demonstrates a design of parallel tubular arcuate peripheral elevations in a fixed position as part of a therapeutic table and does not provide for different shapes, sizes, or configuration that can be used in multiple positions simultaneously. The Johnson invention of 1958 does not demonstrate parallel arcuate peripheral elevations in it's design and has limited application to the sacro-iliac region of the pelvis. The Richardson invention of 1980 is designed to have one continuous convex-concave-convex shaped structure with a linear channel that is intended to be applied to the entire spine for self-use as an alternative to manual manipulation. The Speece invention of 1991 does not demonstrate parallel arcuate peripheral elevations in it's design. The Sexton invention of 1991 stipulates that it is for individual home-use and not intended or designed for therapeutic use of professionals. The Meilus invention of 2000 does have parallel peripheral elevations, but has sharp pointed edges designed for self-treatment of muscular therapy that could not be used in conjunction with manual manipulation, because of the patient injury potential. The Saber invention of 2000 is primarily designed to have one continuous convex-concave-convex shaped structure with a linear channel that is intended to be applied to the entire spine with only round peripheral elevated surfaces.

### SUMMARY

In accordance with the concept of the first embodiment is a portable device consisting of two parallel peripheral elevations with either a flat or convex patient contact surface mounted on a rigid planar base with a channel therebetween designed to be used in conjunction with the methods, techniques, and procedures of spinal manual manipulation and manipulative surgery by a doctor educated and trained in the professions of either chiropractic, osteopathy, naturopathy, and physical medicine for the treatment of spinal and pelvic pain disorders, conditions, and syndromes.

### DRAWINGS

### Figures

In the drawings, closely related figures have the same number but different alphabetic suffixes.

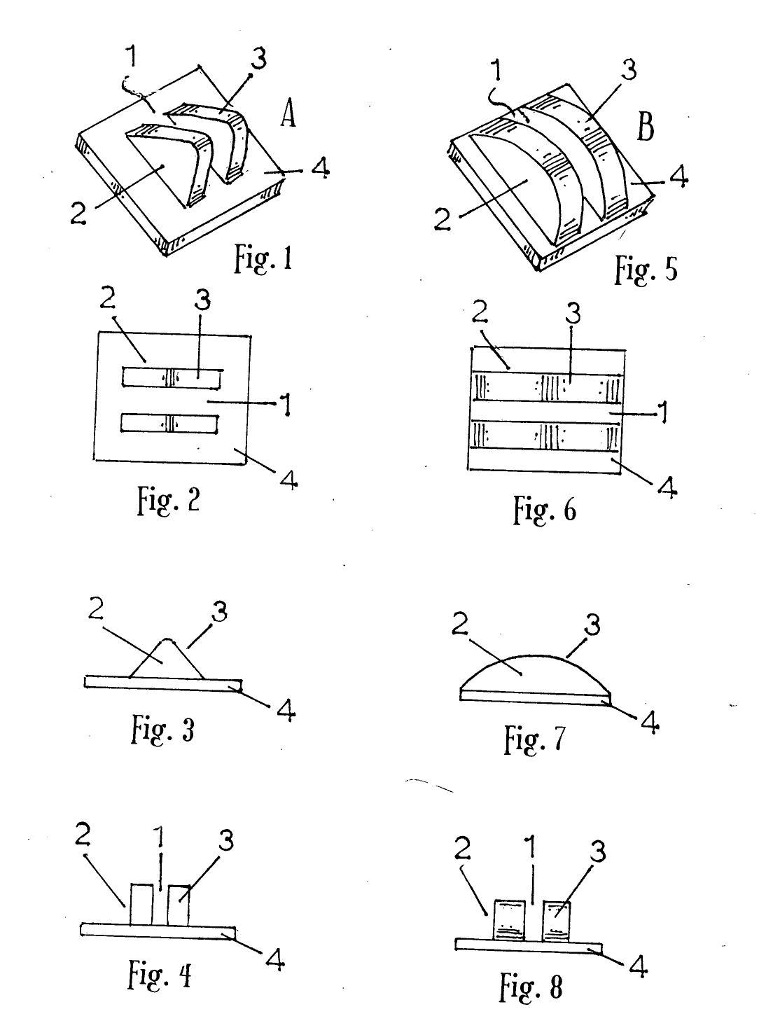

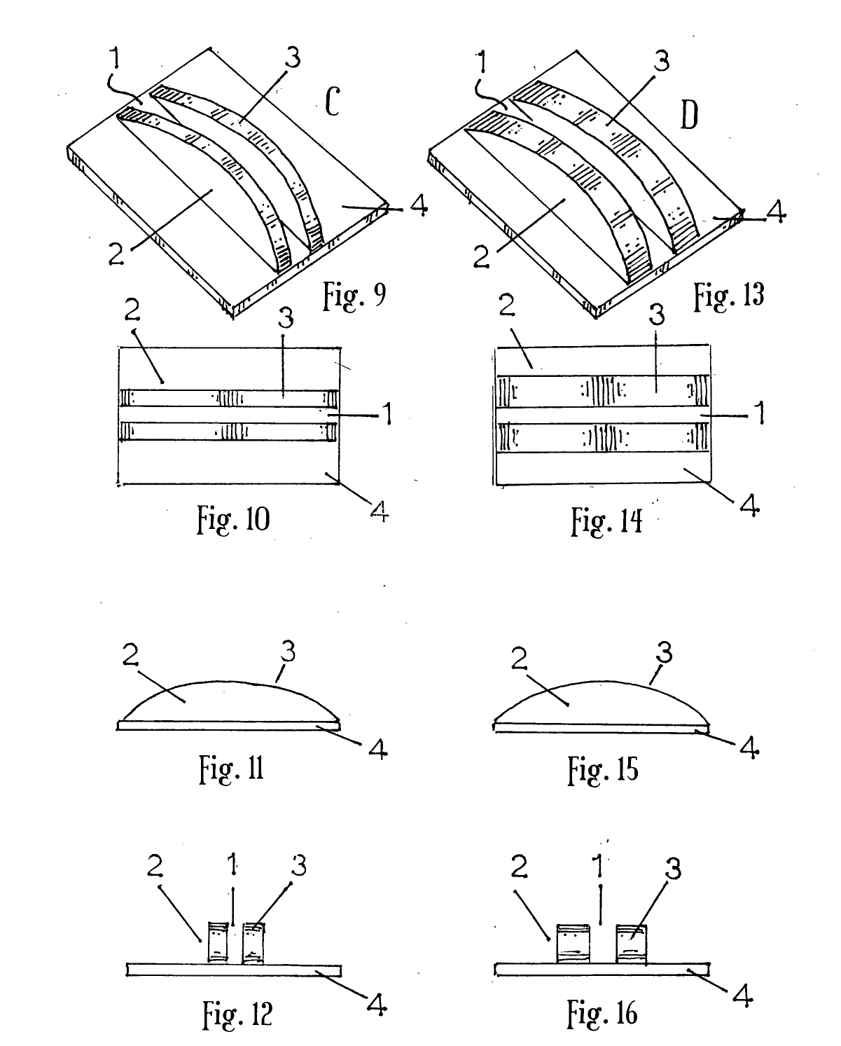

FIG. 1 shows a perspective view of spinal manipulation board A.

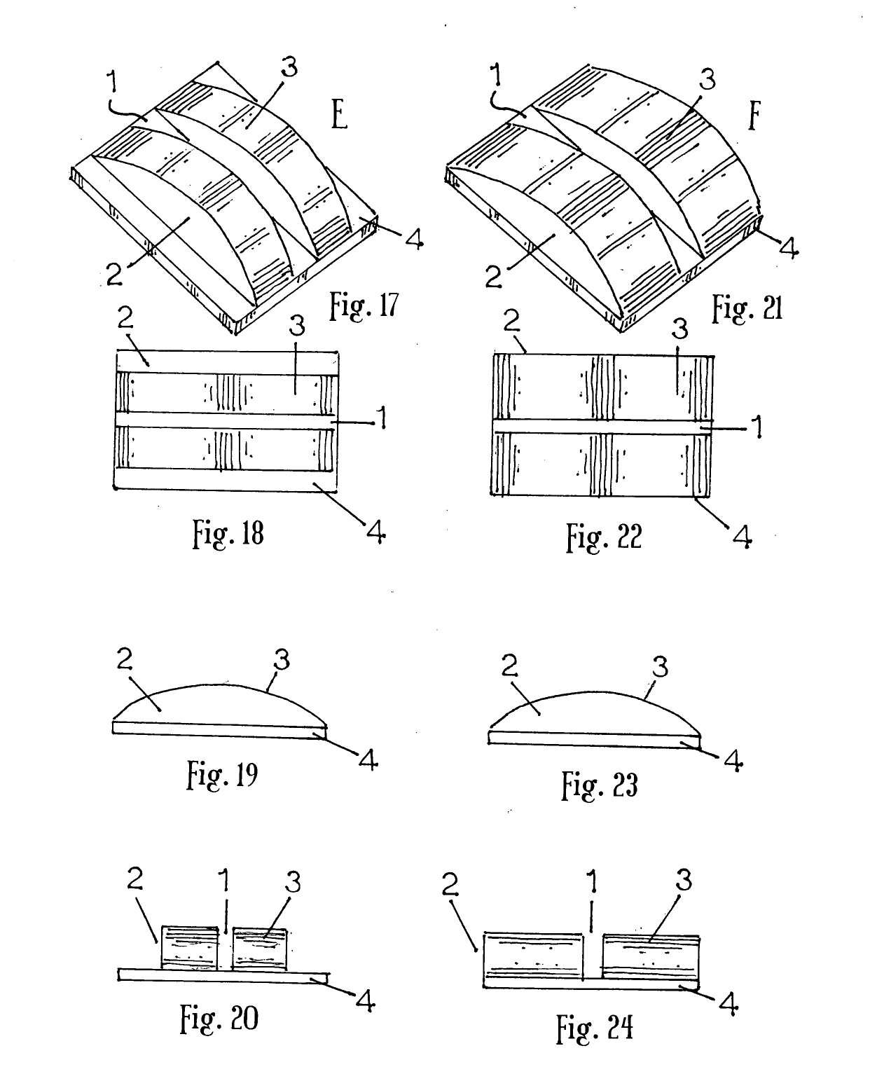

FIG. 2 shows a top sectional view of FIG. 1.

FIG. 3 shows a side sectional view of FIG. 1.

FIG. 4 shows rear sectional view of FIG. 1.

FIG. 5 shows a perspective view of spinal manipulation board B.

FIG. 6 shows a top sectional view of FIG. 5.

FIG. 7 shows a side sectional view of FIG. 5.

FIG. 8 shows rear sectional view of FIG. 5.

FIG. 9 shows a perspective view of spinal manipulation board C.

FIG. 10 shows a top sectional view of FIG. 9.

FIG. 11 shows a side sectional view of FIG. 9.

FIG. 12 shows rear sectional view of FIG. 9.

FIG. 13 shows a perspective view of spinal manipulation board D.

FIG. 14 shows a top sectional view of FIG. 13.

FIG. 15 shows a side sectional view of FIG. 13.

FIG. 16 shows rear sectional view of FIG. 13.

FIG. 17 shows a perspective view of spinal manipulation board E.

FIG. 18 shows a top sectional view of FIG. 17.

FIG. 19 shows a side sectional view of FIG. 17.

FIG. 20 shows rear sectional view of FIG. 17.

FIG. 21 shows a perspective view of spinal manipulation board F.

FIG. 22 shows a top sectional view of FIG. 21.

FIG. 23 shows a side sectional view of FIG. 21.

FIG. 24 shows rear sectional view of FIG. 21.

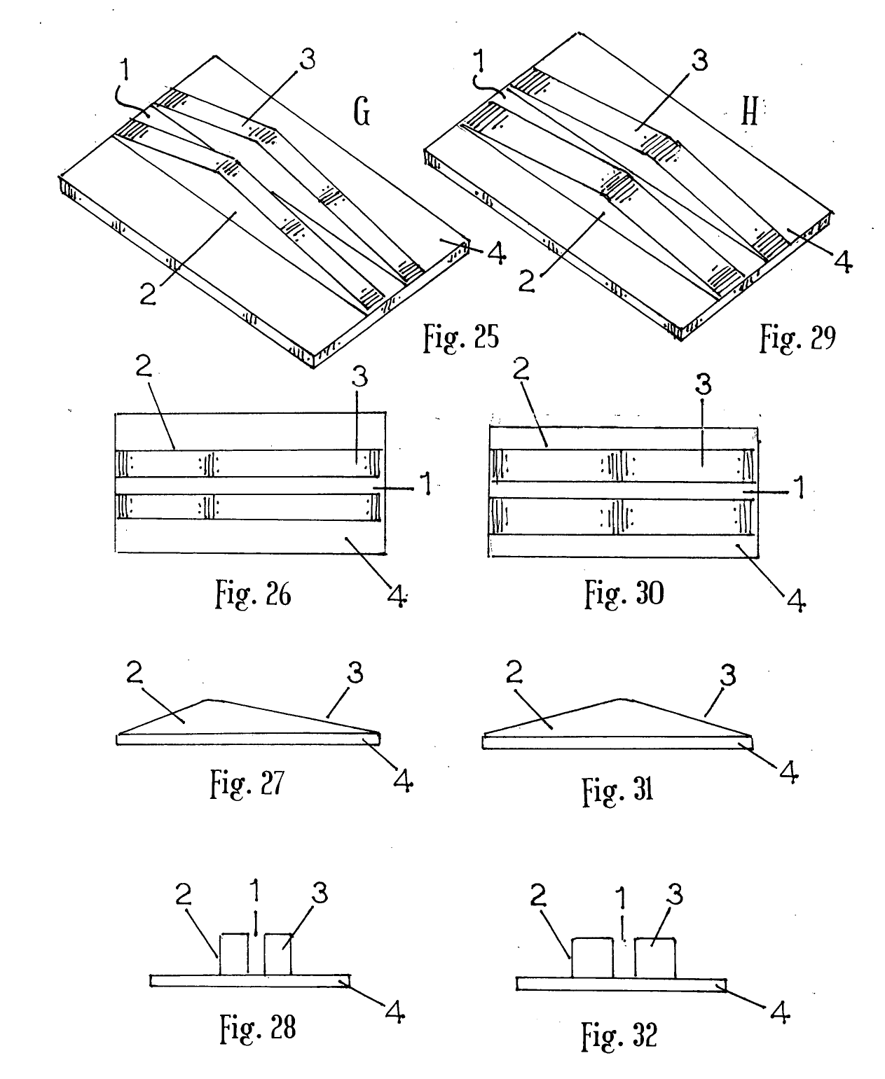

FIG. 25 shows a perspective view of spinal manipulation board G.

FIG. 26 shows a top sectional view of FIG. 25.

FIG. 27 shows a side sectional view of FIG. 25.

FIG. 28 shows rear sectional view of FIG. 25.

FIG. 29 shows a perspective view of spinal manipulation board H.

FIG. 30 shows a top sectional view of FIG. 29.

FIG. 31 shows a side sectional view of FIG. 29.

FIG. 32 shows rear sectional view of FIG. 29.

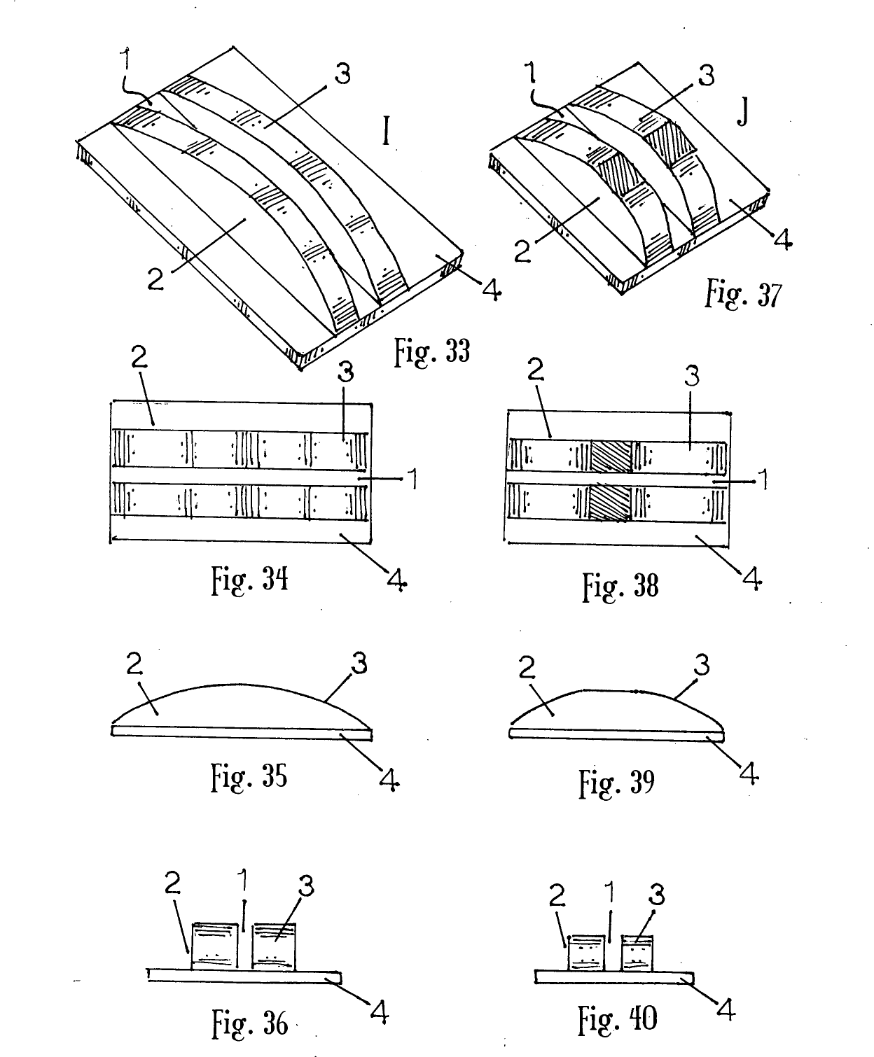

FIG. 33 shows a perspective view of spinal manipulation board I.

FIG. 34 shows a top sectional view of FIG. 33.

FIG. 35 shows a side sectional view of FIG. 33.

FIG. 36 shows rear sectional view of FIG. 33.

FIG. 37 shows a perspective view of spinal manipulation board J.

FIG. 38 shows a top sectional view of FIG. 37.

FIG. 39 shows a side sectional view of FIG. 37.

FIG. 40 shows rear sectional view of FIG. 37.

### DRAWINGS

### Reference Letters

A shows a variation of the first embodiment by design with parallel triangular peripheral elevations with minimum width contact surfaces rounded at vertical apex.
B shows a variation of the first embodiment by design with short parallel arcuate peripheral elevations with medium width contact surfaces.
C shows a variation of the first embodiment by design with full length parallel arcuate peripheral elevations with a minimum width contact surfaces.
D shows a variation of the first embodiment by design with full length parallel arcuate peripheral elevations with a medium width contact surfaces.
E shows a variation of the first embodiment by design with full length parallel arcuate peripheral elevations with a large width contact surfaces.
F shows a variation of the first embodiment by design with full length parallel arcuate peripheral elevations with a maximum width contact surfaces.
G shows a variation of the first embodiment by design with full length parallel unequal triangular peripheral elevations with a minimum width contact surfaces rounded at vertical apex.
H shows a variation of the first embodiment by design with full length parallel triangular peripheral elevations with a medium width contact surfaces rounded at vertical apex.
I shows a variation of the first embodiment by design with long length parallel arcuate peripheral elevations with a large width contact surfaces.
J shows a variation of the first embodiment by design with full length parallel arcuate peripheral elevations which includes a flat area at the vertical apex with a medium width contact surfaces.

### DRAWINGS

### Reference Numbers

1 Channel between parallel peripheral elevations.
2 Parallel peripheral elevations.
3 Contact surface of peripheral elevation.
4 Rigid planar base.

### DETAILED DESCRIPTION OF PREFERRED EMBODIMENTS

The concept of the first embodiment is a portable device consisting of two parallel peripheral elevations with either flat or convex contact surfaces mounted on a rigid planar base with a channel therebetween constructed of any rigid or semi-rigid natural or man-made material such as wood, metal, rubber or plastic, or a combination thereof, sufficient in stability and density to be used in conjunction with the methods, techniques, and procedures of manual manipulation and manipulative surgery.

FIG. 1 is a perspective view of spinal manipulation board A, which is a variation of the first embodiment by design with parallel triangular peripheral elevations with minimum width contact surfaces rounded at vertical apex. FIG. 1 includes the concept of the first embodiment of 1 a channel between the parallel peripheral elevations, 2 two parallel peripheral elevations, 3 contact surface of peripheral elevation, and 4 rigid planar base. FIG. 2 is a top view of spinal manipulation board A, which includes the concept of the first embodiment of 1 a channel between the parallel peripheral elevations, 2 two parallel peripheral elevations, 3 contact surface of peripheral elevation, and 4 rigid planar base. FIG. 3 is a side view of spinal manipulation board A, which includes the concept of the first embodiment of 1 a channel between the parallel peripheral elevations, 2 two parallel peripheral elevations, 3 contact surface of peripheral elevation, and 4 rigid planar base. FIG. 4 is a rear view of spinal manipulation board A, which includes the concept of the first embodiment of 1 a channel between the parallel peripheral elevations, 2 two parallel peripheral elevations, 3 contact surface of peripheral elevation, and 4 rigid planar base.

FIG. 5 is a perspective view of spinal manipulation board B, which is a variation of the first embodiment by design with short parallel arcuate peripheral elevations with medium width contact surfaces. FIG. 5 includes the concept of the first embodiment of 1 a channel between the parallel peripheral elevations, 2 two parallel peripheral elevations, 3 contact surface of peripheral elevation, and 4 rigid planar base. FIG. 6 is a top view of spinal manipulation board B, which includes the concept of the first embodiment of 1 a channel between the parallel peripheral elevations, 2 two parallel peripheral elevations, 3 contact surface of peripheral elevation, and 4 rigid planar base. FIG. 7 is a side view of spinal manipulation board B, which includes the concept of the first embodiment of 1 a channel between the parallel peripheral elevations, 2 two parallel peripheral elevations, 3 contact surface of peripheral elevation, and 4 rigid planar base. FIG. 8 is a rear view of spinal manipulation board B, which includes the concept of the first embodiment of 1 a channel between the parallel peripheral elevations, 2 two parallel peripheral elevations, 3 contact surface of peripheral elevation, and 4 rigid planar base.

FIG. 9 is a perspective view of spinal manipulation board C, which is a variation of the first embodiment by design with full length parallel arcuate peripheral elevations with a minimum width contact surfaces. FIG. 9 includes the concept of the first embodiment of 1 a channel between the parallel peripheral elevations, 2 two parallel peripheral elevations, 3 contact surface of peripheral elevation, and 4 rigid planar base. FIG. 10 is a top view of spinal manipulation board C, which includes the concept of the first embodiment of 1 a channel between the parallel peripheral elevations, 2 two parallel peripheral elevations, 3 contact surface of peripheral elevation, and 4 rigid planar base. FIG. 11 is a side view of spinal manipulation board C, which includes the concept of the first embodiment of 1 a channel between the parallel peripheral elevations, 2 two parallel peripheral elevations, 3 contact surface of peripheral elevation, and 4 rigid planar base. FIG. 12 is a rear view of spinal manipulation board C, which includes the concept of the first embodiment of 1 a channel between the parallel peripheral elevations, 2 two parallel peripheral elevations, 3 contact surface of peripheral elevation, and 4 rigid planar base.

FIG. 13 is a perspective view of spinal manipulation board D, which is a variation of the first embodiment by design with full length parallel arcuate peripheral elevations with a medium width contact surfaces. FIG. 13 includes the concept of the first embodiment of 1 a channel between the parallel peripheral elevations, 2 two parallel peripheral elevations, 3 contact surface of peripheral elevation, and 4 rigid planar base. FIG. 14 is a top view of spinal manipulation board D, which includes the concept of the first embodiment of 1 a channel between the parallel peripheral elevations, 2 two parallel peripheral elevations, 3 contact surface of peripheral elevation, and 4 rigid planar base. FIG. 15 is a side view of spinal manipulation board D, which includes the concept of the first embodiment of 1 a channel between the parallel peripheral elevations, 2 two parallel peripheral elevations, 3 contact surface of peripheral elevation, and 4 rigid planar base. FIG. 16 is a rear view of spinal manipulation board D, which includes the concept of the first embodiment of 1 a channel between the parallel peripheral elevations, 2 two parallel peripheral elevations, 3 contact surface of peripheral elevation, and 4 rigid planar base.

FIG. 17 is a perspective view of spinal manipulation board E, which is a variation of the first embodiment by design with full length parallel arcuate peripheral elevations with a large width contact surfaces. FIG. 17 includes the concept of the first embodiment of 1 a channel between the parallel peripheral elevations, 2 two parallel peripheral elevations, 3 contact surface of peripheral elevation, and 4 rigid planar base. FIG. 18 is a top view of spinal manipulation board E, which includes the concept of the first embodiment of 1 a channel between the parallel peripheral elevations, 2 two parallel peripheral elevations, 3 contact surface of peripheral elevation, and 4 rigid planar base. FIG. 19 is a side view of spinal manipulation board E, which includes the concept of the first embodiment of 1 a channel between the parallel peripheral elevations, 2 two parallel peripheral elevations, 3 contact surface of peripheral elevation, and 4 rigid planar base. FIG. 20 is a rear view of spinal manipulation board E, which includes the concept of the first embodiment of 1 a channel between the parallel peripheral elevations, 2 two parallel peripheral elevations, 3 contact surface of peripheral elevation, and 4 rigid planar base.

FIG. 21 is a perspective view of spinal manipulation board F, which is a variation of the first embodiment by design with full length parallel arcuate peripheral elevations with a maximum width contact surfaces. FIG. 21 includes the concept of the first embodiment of 1 a channel between the parallel peripheral elevations, 2 two parallel peripheral elevations, 3 contact surface of peripheral elevation, and 4 rigid planar base. FIG. 22 is a top view of spinal manipulation board F, which includes the concept of the first embodiment of 1 a channel between the parallel peripheral elevations, 2 two parallel peripheral elevations, 3 contact surface of peripheral elevation, and 4 rigid planar base. FIG. 23 is a side view of spinal manipulation board F, which includes the concept of the first embodiment of 1 a channel between the parallel peripheral elevations, 2 two parallel peripheral elevations, 3 contact surface of peripheral elevation, and 4 rigid planar base. FIG. 24 is a rear view of spinal manipulation board F, which includes the concept of the first embodiment of 1 a channel between the parallel peripheral elevations, 2 two parallel peripheral elevations, 3 contact surface of peripheral elevation, and 4 rigid planar base.

FIG. 25 is a perspective view of spinal manipulation board G, which is a variation of the first embodiment by design with full length parallel unequal triangular peripheral elevations with a minimum width contact surfaces rounded at vertical apex. FIG. 25 includes the concept of the first embodiment of 1 a channel between the parallel peripheral elevations, 2 two parallel peripheral elevations, 3 contact surface of peripheral elevation, and 4 rigid planar base. FIG. 26 is a top view of spinal manipulation board G, which includes the concept of the first embodiment of 1 a channel between the parallel peripheral elevations, 2 two parallel peripheral elevations, 3 contact surface of peripheral elevation, and 4 rigid planar base. FIG. 27 is a side view of spinal manipulation board G, which includes the concept of the first embodiment of 1 a channel between the parallel peripheral elevations, 2 two parallel peripheral elevations, 3 contact surface of peripheral elevation, and 4 rigid planar base. FIG. 28 is a rear view of spinal manipulation board G, which includes the concept of the first embodiment of 1 a channel between the parallel peripheral elevations, 2 two parallel peripheral elevations, 3 contact surface of peripheral elevation, and 4 rigid planar base.

FIG. 29 is a perspective view of spinal manipulation board H, which is a variation of the first embodiment by design with full length parallel triangular peripheral elevations with a medium width contact surfaces rounded at vertical apex. FIG. 29 includes the concept of the first embodiment of 1 a channel between the parallel peripheral elevations, 2 two parallel peripheral elevations, 3 contact surface of peripheral elevation, and 4 rigid planar base. FIG. 2 is a top view of spinal manipulation board H, which includes the concept of the first embodiment of 1 a channel between the parallel peripheral elevations, 2 two parallel peripheral elevations, 3 contact surface of peripheral elevation, and 4 rigid planar base. FIG. 3 is a side view of spinal manipulation board H, which includes the concept of the first embodiment of 1 a channel between the parallel peripheral elevations, 2 two parallel peripheral elevations, 3 contact surface of peripheral elevation, and 4 rigid planar base. FIG. 4 is a rear view of spinal manipulation board H, which includes the concept of the first embodiment of 1 a channel between the parallel peripheral elevations, 2 two parallel peripheral elevations, 3 contact surface of peripheral elevation, and 4 rigid planar base.

FIG. 33 is a perspective view of spinal manipulation board I, which is a variation of the first embodiment by design with long length parallel arcuate peripheral elevations with a large width contact surfaces. FIG. 33 includes the concept of the first embodiment of 1 a channel between the parallel peripheral elevations, 2 two parallel peripheral elevations, 3 contact surface of peripheral elevation, and 4 rigid planar base. FIG. 34 is a top view of spinal manipulation board I, which includes the concept of the first embodiment of 1 a channel between the parallel peripheral elevations, 2 two parallel peripheral elevations, 3 contact surface of peripheral elevation, and 4 rigid planar base. FIG. 35 is a side view of spinal manipulation board I, which includes the concept of the first embodiment of 1 a channel between the parallel peripheral elevations, 2 two parallel peripheral elevations, 3 contact surface of peripheral elevation, and 4 rigid planar base. FIG. 36 is a rear view of spinal manipulation board I, which includes the concept of the first embodiment of 1 a channel between the parallel peripheral elevations, 2 two parallel peripheral elevations, 3 contact surface of peripheral elevation, and 4 rigid planar base.

FIG. 37 is a perspective view of spinal manipulation board J, which is a variation of the first embodiment by design with full length parallel arcuate peripheral elevations which includes a flat area at the vertical apex with a medium width contact surfaces. FIG. 37 includes the concept of the first embodiment of 1 a channel between the parallel peripheral elevations, 2 two parallel peripheral elevations, 3 contact surface of peripheral elevation, and 4 rigid planar base. FIG. 38 is a top view of spinal manipulation board J, which includes the concept of the first embodiment of 1 a channel between the parallel peripheral elevations, 2 two parallel peripheral elevations, 3 contact surface of peripheral elevation, and 4 rigid planar base. FIG. 39 is a side view of spinal manipulation board J, which includes the concept of the first embodiment of 1 a channel between the parallel peripheral elevations, 2 two parallel peripheral elevations, 3 contact surface of peripheral elevation, and 4 rigid planar base. FIG. 40 is a rear view of spinal manipulation board J, which includes the concept of the first embodiment of 1 a channel between the parallel peripheral elevations, 2 two parallel peripheral elevations, 3 contact surface of peripheral elevation, ad planar base.

While the above description contains many specifications, these should not be construed as limitations on the scope of the concept of the first embodiment or the variations thereof, but as exemplifications of the presently preferred embodiments described herein. Many other ramifications and variations are possible within the scope of the concept of the first embodiment. Such variations of the concept of the first embodiment could include design of shape, size, configuration, height of parallel peripheral elevations, width of channel between peripheral elevations, width and shape of peripheral elevation contact surfaces, contact surface modifications for comfort of patient, or modifications to enhance the therapeutic effect when used in conjunction with manual manipulation or manipulative surgery. Even though the concept of the first embodiment stipulates intended use by a doctor educated and trained in the methods, techniques, and procedures of manual manipulation and manipulative surgery this should not be interpreted as a restriction for individual home-use or self-use by prescription for a patient as part of treatment protocol.

Thus the scope of the invention should be determined by the appended claims and their legal equivalents, and not by the examples given.

1. A spinal manipulation board consisting of
a. Base.
b. Parallel peripheral elevations.
c. Channel between peripheral elevations to accommodate spinous process anatomy.
d. Both peripheral elevations have contact surfaces.
e. The peripheral elevations slope downward from a vertex to the base.
f. The shape of peripheral elevations is either arcuate, radial, or triangular.
g. The peripheral elevations are convex to the base.
h. Portable.

2. The concept of the first embodiment of claim 1 is a portable device consisting of two parallel peripheral elevations with either flat or convex patient contact surfaces mounted on a rigid planar base with a channel in between to be used in conjunction with the methods, techniques, and procedures of manual manipulation and manipulative surgery.

3. A device of claim 1 to be used by a doctor educated and trained in the professions of either chiropractic, osteopathy, naturopathy, or physical medicine.

4. A device of claim 1 to be positioned on the back of a patient lying supine, sitting, or standing supported by either a table, chair, or wall.

5. The variation to the shape, size, and configuration of the first embodiment of claim 1 is based on the intended therapeutic purpose when used in conjunction with manual manipulation or manipulative surgery.

6. A device of claim 1 that can be used in conjunction with a resisted-assisted-stabilized method of manual manipulation wherein said device provides for a stabilized platform in substitution of the hand and prevents arthrodial recoil.

7. A device of claim 6 that functions as a “third hand” and allows the doctor to use both anatomical hands to perform methods, techniques, and procedures of manual manipulation or manipulative surgery that would otherwise not be possible.

8. A function of claim 7 that can produce biomechanical forces and leverage that the anatomical hand cannot create, because of the shape, size, and configuration of device of claim 1.

9. A function of claim 8 wherein a kinetic chain effect can be produced into the spine by the application of the four basic manual forces of high velocity-high amplitude, high velocity-low amplitude, low velocity-high amplitude, or low velocity-low amplitude being induced into the human body while positioned on the device of claim 1.

10. A function of claim 8 in which the shape, size, and configuration of the device of claim 1 allows for the use of a static vector, transitional vector, translation vector, or axial vector.

11. A function of claim 8 that produces a convex-convex force into a stabilized vertebral motion segment of a kyphotic curve that can produce decompression of an intravertebral disc as a result of the application of either momentary or sustained anterior-posterior pressure.

12. A function of claim 8 in which convex-convex force is applied to two kyphotic curves allows for the decompression of an intravertebral disc in the lordotic curve in between as a result of the application of either momentary or sustained anterior-posterior pressure.

13. A function of the device of claim 1 whereas through the use of such a device produces relaxation of the paravertebral musculature that can be induced either before, during, or after the application of the methods, techniques, or procedures of manual manipulation or manipulative surgery avoiding the use of heat, which can produce a non-specific inflammatory response that can complicate treatment and recovery.

14. A function of the device of claim 1 with consideration of claim 8 allows for the potential of rehabilitation exercises to be performed on said device under the direction and supervision of the doctor.
